(19) United States
(12) Patent Application Publication
(10) Pub. No.: US 2006/0106037 A1
(43) Pub. Date: May 18, 2006

(54) Purin-6-one-derivatives

(76) Inventors: Thomas Bar, Reichenau (DE); Hermann Tenor, Radolfzell (DE); Armin Hatzelmann, Konstanz (DE)

Correspondence Address: NATH & ASSOCIATES PLLC, 112 South West Street, Alexandria, VA, 22314, US

(21) Appl. No.: 10/547,156

(22) PCT Filed: Mar. 2, 2004
(86) PCT No.: PCT/EP04/50234

§ 371 Date: Nov. 3, 2005

(30) Foreign Application Priority Data
Mar. 4, 2003 (EP) 03004740.1

(51) Int. Cl. A61K 31/522; C07D 473/02

(52) U.S. Cl. 514/263.2; 514/263.21; 514/263.23; 544/276

(57) ABSTRACT

The compounds of a certain formula (I) in which e.g. R2, R3, R4 and R5 have the meanings as given below are novel effective PDE2 inhibitors. R2 is hydrogen, 1-4C-alkyl, 1-hydroxy-2-4C-alkyl, 1-4C-alkylcarbonyl or 1-(acetyloxy)-2-4C-alkyl and R3 is Arylbutyl, Heteroarylbutyl, Arylpropyl, Heteroarylpropyl, Arylethyl or Heteroarylethyl; wherein Aryl is phenyl, naphthalenyl or indanyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl, Heteroaryl is pyridinyl, pyrazinyl, pyridazinyl, pyrimidinyl, quinazolinyl, quinoxalinyl, cinnolinyl, quinolyl, isoquinolyl, naphthyridinyl, phthalazinyl, indolyl, isoindolyl, indazolyl, purinyl, pteridinyl, benzofuranyl, benzoxazolyl, benzothiazolyl, benzimidazolyl, oxazolyl, isoxazolyl, thiazolyl, isothiazolyl, imidazolyl, pyrrolyl, pyrazolyl, furanyl or thiophenyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl, R4 is 1-4C-alkoxy which is completely or predominantly substituted by fluorine and R5 is halogen, hydroxyl, nitro, trifluoromethyl, carboxyl, 1-4C-alkyl, 1-4C-alkoxy, 1-4C-alkoxy which is completely or predominantly substituted by fluorine, 1-4C-alkoxycarbonyl, amino, mono- or di-1-4C-alkylamino, aminocarbonyl, mono- or di-1-4C-alkylaminocarbonyl, 1-4C-alkylcarbonylamino, 1-4C-alkylcarbonyloxy, 1-4C-alkylsulfonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, phenylsulfonylamino, phenylsulfonylamino substituted in the phenyl moiety by R10 and/or R11, benzylsulfonylamino or benzylsulfonylamino substituted in the phenyl moiety by R12 and/or R13.

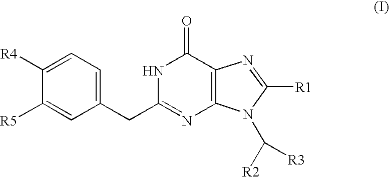

### FIELD OF APPLICATION OF THE INVENTION

The invention relates to novel purin-6-one derivatives, which are used in the pharmaceutical industry for the production of pharmaceutical composition.

### KNOWN TECHNICAL BACKGROUND

In the European patent application EP 0771799, the International patent application WO98/40384 and in the U.S. Pat. No. 5,861,396 purin-6-one derivatives are described as suitable for the treatment of cardiovascular disorders, disorders of the vascular system and of the urogenital system. In the International patent application WO9832755 purindione derivatives are described as phosphodiesterase inhibitors. In the International patent application WO02/50078 substituted imidazotriazones are described as PDE2 inhibitors. In the International patent application WO02/09713 and in the German patent application DE 10108752 A1 selective PDE2 inhibitors are described as medicaments for improving cognition.

### DESCRIPTION OF THE INVENTION

It has now been found that the purin-6-one derivatives, which are described in greater details below, have surprising and particularly advantageous properties.

The invention thus relates to compounds of formula 1 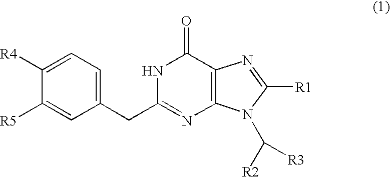
in which
R1 is hydrogen, 1-4C-alkyl, phenyl or phenyl-1-4C-alkyl,
   and in which either
R2 is 1-4C-alkyl, 1-hydroxy-2-4C-alkyl, 1-4C-alkylcarbonyl or 1-(acetyloxy)-2-4C-alkyl and
R3 is hydrogen.
or
R2 is hydrogen, 1-4C-alkyl, 1-hydroxy-2-4C-alkyl, 1-4C-alkylcarbonyl or 1-(acetyloxy)-2-4C-alkyl and
R3 is Arylbutyl, Heteroarylbutyl, Arylpropyl, Heteroarylpropyl, Arylethyl or Heteroarylethyl,
   wherein
Aryl is phenyl, naphthalenyl or indanyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl,
Heteroaryl is pyridinyl, pyrazinyl, pyridazinyl, pyrimidinyl, quinazolinyl, quinoxalinyl, cinnolinyl, quinolyl, isoquinolyl, naphthyridinyl, phthalazinyl, indolyl, isoindolyl, indazolyl, purinyl, pteridinyl, benzofuranyl, benzoxazolyl, benzothiazolyl, benzimidazolyl, oxazolyl, isoxazolyl, thiazolyl, isothiazolyl, imidazolyl, pyrrolyl, pyrazolyl, furanyl or thiophenyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl,
   and in which either
R4 is 1-4C-alkoxy which is completely or predominantly substituted by fluorine and
R5 is halogen, hydroxyl, nitro, trifluoromethyl, carboxyl, 1-4C-alkyl, 1-4C-alkoxy, 1-4C-alkoxy which is completely or predominantly substituted by fluorine, 1-4C-alkoxycarbonyl, amino, mono- or di-1-4C-alkylamino, aminocarbonyl, mono- or di-1-4C-alkylaminocarbonyl, 1-4C-alkylcarbonylamino, 1-4C-alkylcarbonyloxy, 1-4C-alkylsulfonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, phenylsulfonylamino, phenylsulfonylamino substituted in the phenyl moiety by R10 and/or R11, benzylsulfonylamino or benzylsulfonylamino substituted in the phenyl moiety by R12 and/or R13,
   or
R4 is halogen, hydroxyl, nitro, trifluoromethyl, carboxyl, 1-4C-alkyl, 1-4C-alkoxy, 1-4C-alkoxycarbonyl, amino, mono- or di-1-4C-alkylamino, aminocarbonyl, mono- or di-1-4C-alkylaminocarbonyl, 1-4C-alkylcarbonylamino, 1-4C-alkylcarbonyloxy, 1-4C-alkylsulfonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, phenylsulfonylamino, phenylsulfonylamino substituted in the phenyl moiety by R10 and/or R11, benzylsulfonylamino or benzylsulfonylamino substituted in the phenyl moiety by R12 and/or R13, and
R5 is 1-4C-alkoxy which is completely or predominantly substituted by fluorine,
R6 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R7 is halogen, 1-4C-alkyl or 1-4C-alkoxy,
R8 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R9 is halogen, 1-4C-alkyl or 1-4C-alkoxy,
R10 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R11 is halogen, 1-4C-alkyl or 1-4C-alkoxy,
R12 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R13 is halogen, 1-4C-alkyl or 1-4C-alkoxy,
   the salts of these compounds, as well as the N-oxides, enantiomers and tautomers of these compounds and their salts.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

Phenyl-1-4C-alkyl stands for one of the abovementioned 1-4C-alkyl radicals, which is substituted by an phenyl radical. Examples which may be mentioned are the phenylpropyl, phenylethyl and the benzyl radical.

1-hydroxy-2-4C-alkyl stands for one of the above-mentioned 2-4C-alkyl radicals, which is substituted in 1-position by a hydroxyl group. Examples which may be mentioned are 1-hydroxyethyl, 1-hydroxypropyl and 1-hydroxybutyl.

1-4C-Alkylcarbonyl is a carbonyl group to which one of the abovementioned 1-4C-alkyl radicals is bonded. An example is the acetyl radical [CH₃C(O)—].

1-4C-Alkylcarbonyloxy stands for a carbonyloxy group to which one of the abovementioned 1-4C-alkyl radicals is bonded. An example is the acetoxy radical [CH₃C(O)—O—].

1-(acetyloxy)-2-4C-alkyl stands for one of the above-mentioned 2-4C-alkyl radicals, which is substituted in 1-position by an acetyloxy group. An example which may be mentioned is 1-(acetyloxy)ethyl.

Halogen within the meaning of the present invention is bromine, chlorine or fluorine.

1-4C-Alkoxy is a radical which, in addition to the oxygen atom, contains a straight-chain or branched alkyl radical having 1 to 4 carbon atoms. Alkoxy radicals having 1 to 4 carbon atoms which may be mentioned in this context are, for example, the butoxy, isobutoxy, sec-butoxy, tert-butoxy, propoxy, isopropoxy, ethoxy and methoxy radicals.

1-4C-Alkoxycarbonyl is a carbonyl group to which one of the abovementioned 1-4C-alkoxy radicals is bonded. Examples are the methoxycarbonyl [CH₃O—C(O)—] and the ethoxycarbonyl [CH₃CH₂O—C(O)—] radical.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy groups are replaced by fluorine atoms.

Mono- or di-1-4C-alkylamino radicals contain in addition to the nitrogen atom, one or two of the abovementioned 1-4C-alkyl radicals. Preferred are the di-1-4C-alkylamino radicals, especially the dimethylamino, the diethylamino and the dipropylamino radical.

Mono- or di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical.

Mono-or di-1-4C-alkylaminosulfonyl stands for a sulfonyl group to which one of the abovementioned mono- or di-1-4C-alkylamino radicals is bonded. Examples which may be mentioned are the methylaminosulfonyl, the dimethylaminosulfonyl and the ethylaminosulfonyl radical.

An 1-4C-alkylcarbonylamino radical is, for example, the propionylamino [C₃H₇C(O)NH—] and the acetylamino radical [CH₃C(O)NH—].

1-4C-Alkylsulfonyl is a sulfonyl group to which one of the abovementioned 1-4C-alkyl radicals is bonded. An example is the methanesulfonyl radical (CH₃SO₂—).

An 1-4C-alkylsulfonylamino radical is, for example, ethylsulfonylamino or the methylsulfonylamino radical.

N-oxide denotes a N-oxide in the purin ring system and/or a N-oxide in any of the mentioned heteroaryl rings containing a nitrogen atom.

Suitable salts for compounds of the formula 1—depending on substitution—are all acid addition salts or all salts with bases. Particular mention may be made of the pharmacologically tolerable inorganic and organic acids and bases customarily used in pharmacy. Those suitable are, on the one hand, water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3hydroxy-2-naphtholic acid, the acids being employed in salt preparation—depending on whether a mono- or polybasic acid is concerned and depending on which salt is desired—in an equimolar quantitative ratio or one differing therefrom.

On the other hand salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds according to the invention on an industrial scale, are converted into pharmacologically tolerable salts by processes known to the person skilled in the art.

According to experts knowledge the compounds of the invention as wet as their salts may contain, e.g. when isolated in crystalline form, varying amounts of solvents, included within the scope of the invention are therefore all solvates and in particular all hydrates of the compounds of formula 1 as well as all solvates and in particular all hydrates of the salts of the compounds of formula 1.

Compounds of formula 1 to be emphasized are those in which
R1 is hydrogen, 1-2C-alkyl, phenyl, phenylethyl or phenylpropyl,
R2 is 1-hydroxy-2-4C-alkyl, 1-4C-alkylcarbonyl or 1-(acetyloxy)-2-4C-alkyl,
R3 is hydrogen, Arylbutyl, Heteroarylbutyl, Arylpropyl, Heteroarylpropyl, Arylethyl or Heteroarylethyl,
   wherein
Aryl is phenyl or naphthalenyl,
Heteroaryl is pyridinyl, pyrimidinyl, thiofuranyl, indolyl or furanyl, and in which either
R4 is 1-4C-alkoxy which is completely or predominantly substituted by fluorine and
R5 is halogen, hydroxyl, nitro, trifluoromethyl, 1-4C-alkyl, 1-4C-alkoxy, 1-4C-alkoxy which is completely or predominantly substituted by fluorine, amino, mono- or di-1-4C-alkylamino, 1-4C-alkylcarbonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9,
   or
R4 is halogen, hydroxyl, nitro, trifluoromethyl, 1-4C-alkyl, 1-4C-alkoxy, amino, mono- or d-1-4C-alkylamino, 1-4C-alkylcarbonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, and
R5 is 1-4C-alkoxy which is completely or predominantly substituted by fluorine,
R6 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R7 is halogen, 1-4C-alkyl or 1-4C-alkoxy,
R8 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R9 is halogen, 1-4C-alkyl or 1-4C-alkoxy,
   the salts of these compounds, as well as the N-oxides, enantiomers and tautomers of these compounds and their salts.

Compounds of formula 1 which are particularly to be emphasized are those in which
R1 is hydrogen, methyl, phenyl, phenylethyl or phenylpropyl,
R2 is 1-hydroxyethyl, acetyl or 1-(acetyloxy)ethyl,
R3 is hydrogen or phenylpropyl,
   and in which either
R4 is difluoromethoxy and
R5 is nitro, amino, methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
   or
R4 is chlorine or methoxy and
R5 is difluoromethoxy,
   the salts of these compounds, as well as the N-oxides, enantiomers and tautomers of these compounds and their salts.

Preferred compounds of formula 1 are those in which
R1 is hydrogen or methyl,
R2 is 1-hydroxyethyl, acetyl or 1-(acetyloxy)ethyl,
R3 is phenylpropyl,
   and in which either
R4 is difluoromethoxy and
R5 is methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
   or
R4 is chlorine or methoxy and
R5 is difluoromethoxy,
   the salts of these compounds, as well as the N-oxides, enantiomers and tautomers of these compounds and their salts.

An embodiment of the preferred compounds of formula I are those in which
R1 is hydrogen or methyl,
R2 is 1-hydroxyethyl, acetyl or 1-(acetyloxy)ethyl,
R3 is phenylpropyl,
   and in which either
R4 is difluoromethoxy and
R5 is methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
   or
R4 is methoxy and
R5 is difluoromethoxy,
   the salts of these compounds, as well as the N-oxides, enantiomers and tautomers of these compounds and their salts.

A special embodiment of the compounds of the present invention include those compounds of formula 1 in which R1 is hydrogen or methyl.

Another special embodiment of the compounds of the present invention include those compounds of formula 1 in which 1:2 is 1-hydroxyethyl or acetyl.

Still another special embodiment of the compounds of the present invention include those compounds of formula 1 in which R1 is hydrogen or methyl and R2 is 1-hydroxyethyl or acetyl.

A further special embodiment of the compounds of the present invention include those compounds of formula 1 in which R1 is hydrogen or methyl, R2 is 1-hydroxyethyl or acetyl and R3 is phenylpropyl.

Another further special embodiment of the compounds of the present invention include those compounds of formula 1 in which R4 is difluoromethoxy and R5 is methoxy or in which R4 is methoxy and R5 is difluoromethoxy.

In those cases, where neither R2 nor R3 represents a hydrogen atom, the compounds of formula 1 are chiral compounds having a chiral center at the carbon atom, to which the substituents R2 and R3 are attached. The compounds of formula 1 can have an additional chiral center in those cases, where R2 represents a 1-hydroxy-2-4C-alkyl or 1-(acetyloxy)-2-4C-alkyl radical. The invention comprises all conceivable pure diastereomers and pure enantiomers and their mixtures in any mixing ratio, including the racemates.

The compounds of formula 1 according to the invention can, for example, be prepared as described in the following reaction schemes.

Reaction scheme 1 exemplary shows the preparation of compounds of formula 1 in which R1, R3, R4 and R5 have the above-mentioned meanings and R2 is 1-hydroxyethyl or acetyl.

Reaction scheme 1: in a first reaction step 2-amino-2-cyanoacetamide is (a) reacted with a compound of formula 5 in which R1 has the above-mentioned meanings and then (b) with a compound of formula 4, in which R3 has the above-mentioned meanings. In a second reaction step the obtained 5amino-4-carboxamide-1H-imidazole derivative of formula 3 is reacted with a phenyl-acetic acid ester derivative of formula 2, in which R4 and R5 have the above-mentioned meanings and R is 1-4C-alkyl to yield a compound of formula 1, in which R1, R3, R4 and R5 have the above-mentioned meanings and R2 is 1-hydroxyethyl.

The compounds of formula 1, in which R1, R3, R4 and R5 have the above-mentioned meanings and R2 is 1-hydroxyethyl can be converted to corresponding compounds in which R2 is acetyl by an oxidation process.

Preferred methods for the oxidation are DMSO based protocols like the Swern or Pfitzner-Moffat oxidation. Other suitable oxidants are the Dess-Martin reagent, MnO₂, Pyridinium chloro chromate (PCC) or other Cr (VI) reagents, DDO, H₂O₂, NaOCl or NBS.

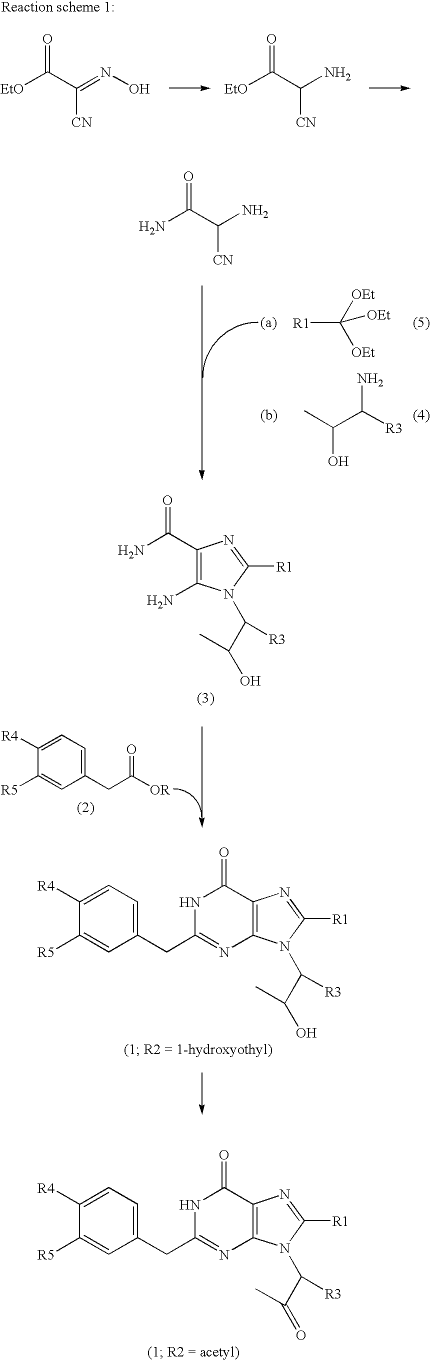

Reaction scheme 2 shows the preparation of compounds of formula 1 in which R1, R3, R4 and R5 have the above-mentioned meanings and is methyl.

Reaction scheme 2: Here, in a first reaction step 2-amino-2-cyanoacetamide is reacted (a) with a compound of formula 5 in which R1 has the above-mentioned meanings and then (b) with a compound of formula 7, in which R3 has the above-mentioned meanings. In a second reaction step the obtained 5-amino-4-carboxamide-1H-imidazole derivative of formula 6 is reacted with a phenyl-acetic acid derivative of formula 2 in which R4 and R5 have the above-mentioned meanings and R is 1-4C-alkyl to yield a compound of formula 1, in which R1, R3, R4 and R5 have the above-mentioned meanings and R2 is methyl.

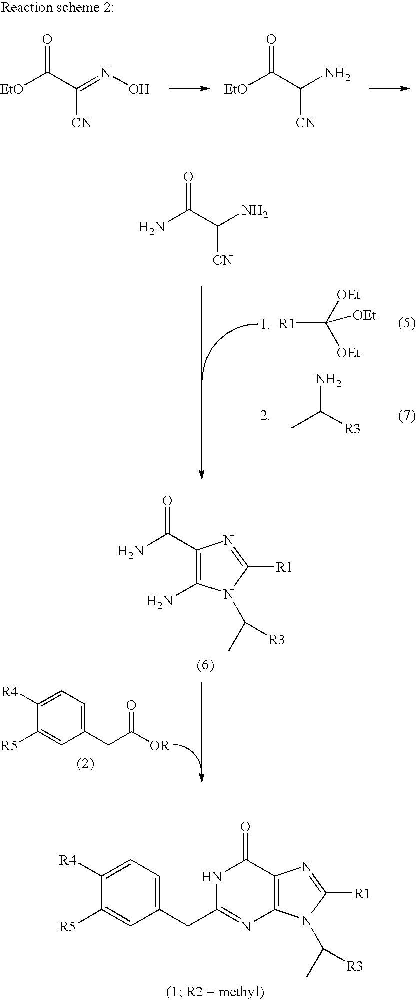

Reaction scheme 3 shows the preparation of certain compounds of formulae 4 and 7.

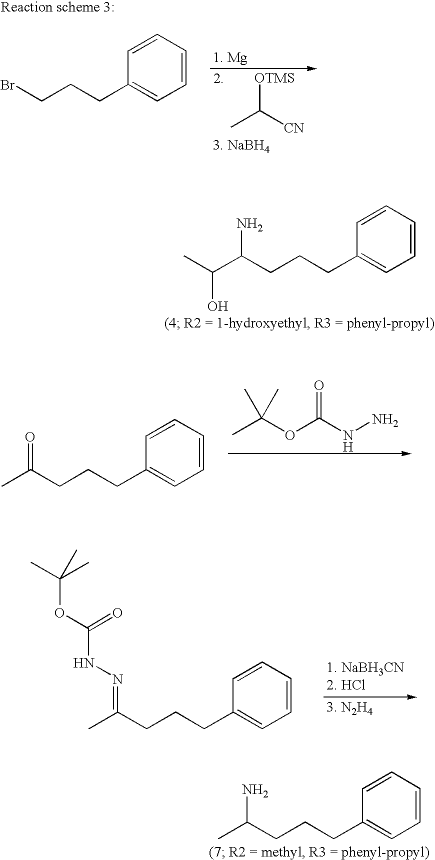

Compounds of the formula 1 obtained can be converted, optionally, into further compounds of formula 1 by derivatization.

For example, from compounds of formula 1, in which

a) R4 or R5 is an ester group, the corresponding acids can be obtained by acidic or alkaline hydrolysis, or the corresponding amides can be prepared by reaction w%nh suitably substituted amines;

b) R4 or R5 is an 1-4C-alkylcarbonyloxy group, the corresponding hydroxyl compounds can be obtained by acidic or alkaline hydrolysis;

c) R4 or R5 is a nitro group, the corresponding amino compounds—which for their part again can be further derivatized—can be obtained by selective catalytic hydrogenation.

The methods mentioned under a), b) and c) are expediently carried out analogously to the methods known to the person skilled in the art.

The compounds of formula 1 can be converted, optionally, into their N-oxides, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

Suitably, the conversions are carried out analogous to methods which are familiar per se to the person skilled in the art, for example, in the manner which is described in the following examples.

The compounds of formulae 2, 4 and 7 are either known or can be prepared in a known manner. Suitable compounds of formula 5, which may be mentioned are triethyl orthoformate, triethyl orthoacetate, triethyl orthobenzoate, trimethyl orthobutyrate, (2,2,2 trimethoxy-ethyl)benzene, (2,2,2 trimethoxy-propyl)benzene and (2,2,2 trimethoxy-butyl)benzene.

2-Amino-2-cyanoacetamide can be prepared starting from ethyl(hydroxyimino)cyanoacetate as described by F. I. Logemann and G. Shaw (Chem. Ind. 1980, 541-542).

It is known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1991.

The substances according to the invention are isolated and purified in a manner known per se, e.g. by distilling off the solvent in vacuo and recrystallizing the residue obtained from a suitable solvent or subjecting it to one of the customary purification methods, such as column chromatography on a suitable support material.

Salts are obtained by dissolving the free compound in a suitable solvent (for example a ketone like acetone, methylethylketone, or methylisobutylketone, an ether, like diethyl ether, tetrahydrofuran or dioxane, a chlorinated hydrocarbon, such as methylene chloride or chloroform, or a low molecular weight aliphatic alcohol, such as ethanol, isopropanol) which contains the desired acid, or to which the desired acid is then added. The salts are obtained by filtering, reprecipitating, precipitating with a non-solvent for the addition salt or by evaporating the solvent. Salts obtained can be converted by basification into the free compounds which, in turn, can be converted into salts. In this manner, pharmacologically non-tolerable salts can be converted into pharmacologically tolerable salts.

The following examples illustrate the invention in greater detail, without restricting it. As well, further compounds of formula 1, of which the preparation is explicitly not described, can be prepared in an analogous way or in a way which is known by a person skilled in the art using customary preparation methods.

In the examples, h stands for hour(s), min for minutes, calc. for calculated, MS for Mass spectrometry and RT for room temperature. The compounds, which are mentioned in the examples as well as their salts are preferred compounds of the invention.

### EXAMPLES

Final Products

### 1. 2-(4-Difluoromethoxy-3-methoxybenzyl)-9-[1-(1-hydroxyethyl)-4-phenylbutyl]-1,9-dihydropurin-6-one

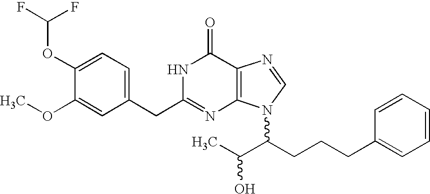

A solution of ethyl(4-difluoromethoxy-3-methoxyphenyl)acetate (3.3 g, 12.6 mmol) and 5-amino-1-[1-(1-hydroxyethyl)-4-phenylbutyl]-1H-imidazole-4-carboxamide (956 mg, 3.2 mmol) in warm ethanol (10 ml) is added to a solution of sodium (363 mg, 15.8 mmol) in ethanol (20 ml), and the mixture is heated under reflux for 16 h. The ethanol is distilled off using a rotary evaporator, the residue is dissolved in dichloromethane (30 ml) and washed with water (20 ml) and most of the dichloromethane is removed using a rotary evaporator. Silica gel chromatography (90 g of silica gel, ethyl acetate/methanol=10/1), recrystallization from ethyl acetate and drying under reduced pressure gives the title compound (0.99 g) as colourless crystals.

MS: calc.: C₂₆H₂₈F₂N₄O₄ (498.21), found: [MH⁺]499.2

### 2. 9-(1-Acetyl-4-phenylbutyl)-2-(4-difluoromethoxy-3-methoxybenzyl)-1,9-dihydropurin-6-one

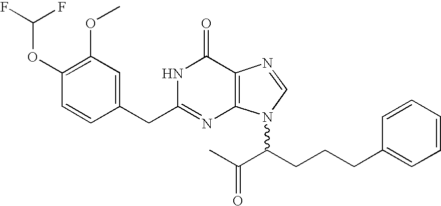

At <5° C., pyridine/sulphur trioxide complex (700 mg, 4.4 mmol) is added to a solution of 2-(4-difluoromethoxy-3-methoxybenzyl)-9-[1-(1-hydroxyethyl)-4-phenylbutyl]-1,9-dihydropurin-6-one (498 mg, 1.0 mmol) and triethylamine (1.39 ml, 10 mmol) in dichloromethane (10 ml) and DMSO (3 ml), and the mixture is stirred at <5° C. for 1 h and then at RT for 15 h. 1N aqueous sodium hydroxide solution (15 ml) added to the solution, the organic phase is separated off and the aqueous phase is extracted twice with dichloromethane (in each case 10 ml). The combined organic phases are dried over magnesium sulphate and the solvent is removed using a rotary evaporator. Silica gel chromatography (50 g of silica gel, ethyl acetate/methanol=10/1) and drying under reduced pressure gives the title compound (470 mg) as a colourless foam.

MS: calc.: C₂₆H₂₆F₂N₄O₄ (496.19), found: [MH⁺]497.2

### 3. 2-(3-Difluoromethoxy-4-methoxybenzyl)-9-[1-(1-hydroxyethyl)-4-phenylbutyl]-1,9-dihydropurin-6-one

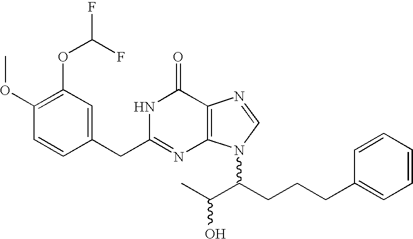

A solution of ethyl(3-difluoromethoxy-4-methoxyphenyl)acetic (3.1 g, 12 mmol) and 5-amino-1-[1-(1-hydroxyethyl)-4-phenylbutyl]-1H-imidazole-4-carboxamide (907 mg, 3 mmol) in warm ethanol (10 ml) is added to a solution of sodium (345 mg, 15 mmol) in ethanol (20 ml), and the mixture is heated under reflux for 16 h. The ethanol is distilled off using a rotary evaporator, the residue is dissolved in dichloromethane (30 ml) and washed with water and most of the dichloromethane is removed using a rotary evaporator. Silica gel chromatography (90 g of silica gel, ethyl acetate/methanol=10/1), recrystallization from ethyl acetate and drying under reduced pressure gives the title compound (1.06 g) as colourless crystals.

MS: calc.: C₂₆H₂₆F₂N₄O₄ (498.21), found: [MH⁺]499.2

### 4. 9-(1-Acetyl-4-phenylbutyl)-2-(3-difluoromethoxy-4-methoxybenzyl)-1,9-dihydropurin-6-one

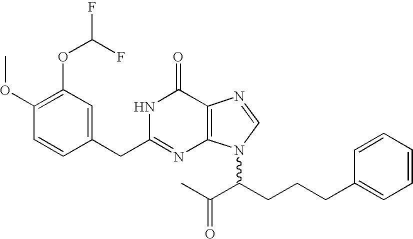

At <5° C., pyridine/sulphur trioxide complex (700 mg, 4.4 mmol) is added to a solution of 2-(3-difluoromethoxy-4-methoxybenzyl)-9-[1-(1-hydroxyethyl)-4-phenylbutyl]-1,9-dihydropurin-6-one (498 mg, 1.0 mmol) and triethylamine (1.39 ml, 10 mmol) in dichloromethane (10 ml) and DMSO (3 ml), and the mixture is stirred at <5° C. for 1 h and then at RT for 15 h. 1 N aqueous sodium hydroxide solution (15 ml) is added to the solution, the organic phase is separated off and the aqueous phase is extracted twice with dichloromethane (in each case 10 ml). The combined organic phases are dried over magnesium sulphate and the solvent is removed using a rotary evaporator. Silica gel chromatography (50 g of silica gel, ethyl acetate/methanol=10/1) and drying under high vacuum gives the title compound (460 mg) as a colourless foam.

### 5. 2-[2-(4-Difluoromethoxy-3-methoxybenzyl)-6-oxo-1,6-dihydropurin-9-yl]-1-methyl-5-phenylphentyl acetate

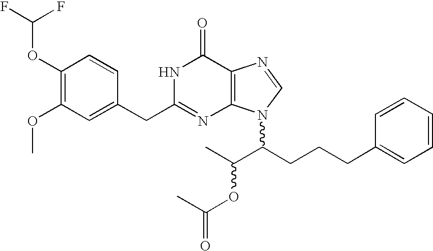

A solution of ethyl(3-difluoromethoxy-4-methoxyphenyl)acetic (3.1 g, 12 mmol) and 5-amino-1-[1-(1-hydroxyethyl)-4-phenylbutyl]-1H-imidazole-4-carboxamide (907 mg, 3 mmol) in warm ethanol (10 ml) is added to a solution of sodium (345 mg, 15 mmol) in ethanol (20 ml), and the mixture is heated under reflux for 16 h. The solvent is removed using a rotary evaporator and the residue is dissolved in ethyl acetate (40 ml). The solution is washed with water (20 ml) and dried over magnesium sulphate, and the solvent is removed using a rotary evaporator. Silica gel chromatography and drying under a high vacuum gives the title compound (210 mg) as a colourless resin. Additionally, 2-(4-difluoromethoxy-3-methoxybenzyl)-9-[1-(1-hydroxyethyl)-4-phenylbutyl]-1,9-dihydropurin-6-one (990 mg) is obtained

MS: calc.: C₂₈H₃₀F₂N₄O₅ (540.22), found: [MH⁺]541.1

### 6. 2-(4-Difluoromethoxy-3-methoxybenzyl)-9-[1-(1-hydroxyethyl)-4-phenylbutyl]-1,9-dihydropurin-6-one

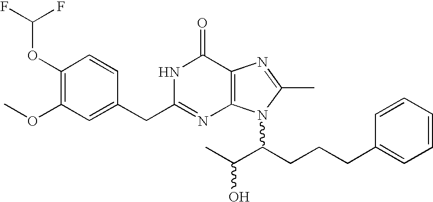

At RT, 4.16 g (16.0 mmol) of ethyl(4-difluoromethoxy-3-methoxyphenyl)acetic and 1.29 g (4.1 mmol) of 5-amino-1-[1-(1-hydroxyethyl)-4-phenylbutyl]-2methyl-1H-imidazole-4-carboxamide 5 ml of warm ethanol are added to a solution of 0.47 g (20.4 mmol) of sodium in 35 ml of absolute ethanol, and the mixture is heated under reflux for 18 h. The ethanol is distilled off using a rotary evaporator, 20 ml of water are added to the residue and the mixture is then extracted 4 times with in each case 50 ml of ethyl acetate. The combined organic phases are dried over magnesium sulphate and the ethyl acetate is then removed using a rotary evaporator. Silica gel chromatography (200 g of silica gel, ethyl acetate/methanol 10:1) gives the title compound (0.560 g) as a white crystalline solid.

MS: calc.: C₂₇H₃₀N₄O₄F₂ (512.22), found: [MH⁺]513.2

### 7. 9-(1-Acetyl-4-phenylbutyl)-2-(4-difluoromethoxy-3-methoxybenzyl)-8-methyl-1,9-dihydropurin-6-one

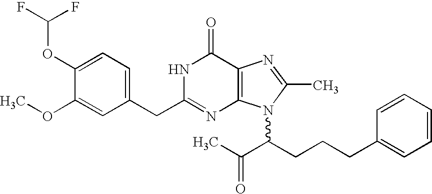

7 ml of absolute dichloromethane and 0.89 ml (6.39 mmol) of triethylamine are added to 0.35 g (0.68 mmol) of 2-(4-difluoromethoxy-3-methoxybenzyl)-9-[1-(1-hydroxyethyl)-4-phenylbutyl]-1,9-dihydropurin-6-one, and the mixture is cooled to 0° C. using an ice-bath. 2.2 ml of DMSO and 0.48 g (3.0 mmol) of pyridine/sulphur trioxide complex are added, and the mixture is then, under an atmosphere of nitrogen, stirred with ice-cooling for 1 h and at RT for a further 15 h. 20 ml of water are added to the solution, and the mixture is extracted three times with in each case 25 ml of dichloromethane. The organic phases are washed with water and then dried over magnesium sulphate and concentrated using a rotary evaporator. Silica gel chromatography (35 g of silica gel, ethyl acetate/methanol=10:1) gives the title compound (0.09 g) as a white crystalline solid.

MS: calc.: C₂₇H₂₆N₄O₄F₂ (510.21), found: [MH⁺]511.2

### 8. 2-(3-Difluoromethoxy-4-methoxybenzyl)-9-[1-(1-hydroxyethyl)-4-phenylbutyl]-8-methyl-1,9-dihydropurin-6-one

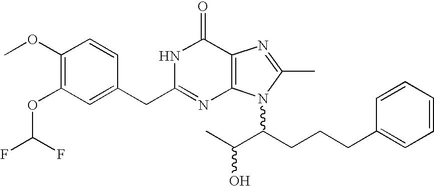

At RT, 2.98 g (11.5 mmol) of ethyl(3-difluoromethoxy-4-methoxyphenyl)acetic and 0.91 g (2.9 mmol of 5-amino-1-[1-(1-hydroxyethyl)-4-phenylbutyl]-2-methyl-1H-imidazole-4-carboxamide in 15 ml of warm ethanol are added to a solution of 0.34 g (14.5 mmol) of sodium in 15 ml of absolute ethanol, and the mixture is heated under reflux for 18 h. The ethanol is distilled off using a rotary evaporator, 20 ml of water are added to the residue and the mixture is then extracted 5 times with in each case 30 ml of ethyl acetate. The combined organic phases are dried over magnesium sulphate and the ethyl acetate is then removed using a rotary evaporator. Silica gal chromatography (120 g of silica gel, ethyl acetate/methanol=10:1) gives the tile compound (0.29 g) as a slightly yellowish foam.

MS: calc.: C₂₇H₃₀N₄O₄F₂ (512.22), found: [MH⁺]513.2

### 9. 9-(1-Acetyl-4-phenylbutyl)-2-(3-difluoromethoxy-4-methoxybenzyl)-8-methyl-1,9-dihydropurin-6-one

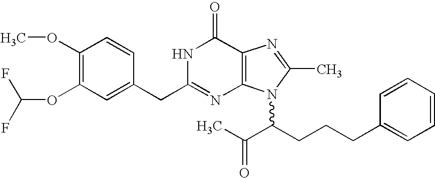

0.20 g (0.39 mmol) of 2-(3-difluoromethoxy-4-methoxybenzyl)-9-[1-(1-hydroxyethyl)-4-phenylbutyl]-8-methyl-1,9-dihydropurin-6-one is dissolved in 4.3 ml of dichloromethane, 0.57 ml (4.1 mmol) of triethylamine is added and the mixture is cooled to 0° C. Using an ice bath. 1.3 ml of DMSO and 0.30 g (1.9 mmol) of pyridine/sulphur trioxide complex are added, and the mixture is then, under an atmosphere of nitrogen, stirred for 1 h with ice-cooling and at RT for a further 15 h. 20 ml of water are then added, and the mixture is extracted 3 times with in each case 25 ml of dichloromethane. The organic phases are washed with water and then dried over magnesium sulphate and concentrated using a rotary evaporator. Silica gel chromatography (25 g of silica gel, ethyl acetate/methanol=10:1) gives the title compound (0.16 g) as a yellow oil.

MS: calc.: C₂₇H₂₆N₄O₄F₂ (510.21), found: [MH⁺]511.2

### 10. 2-(4-Difluoromethoxy-3-methoxybenzyl)-9-(2-hydroxypropyl)-1,9-dihydropurin-6-one

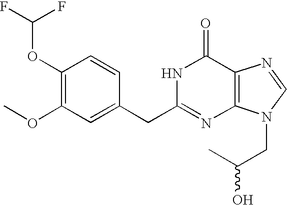

At RT, 2.3 g (8.8 mmol) of ethyl(4-difluoromethoxy-3-methoxyphenyl)acetate and 0.46 g (2.5 mmol) of 5-amino-1-(2-hydroxypropyl)-1H-imidazole-4-carboxamide in 5 ml of warm ethanol are added to a solution of 0.52 g (11.0 mmol) of sodium in 20 ml of absolute ethanol, and the mixture is heated under reflux for 18 h. The ethanol is distilled off using a rotary evaporator, 20 ml of water are added to the residue and the mixture is neutralized using 2N acetic acid. Precipitated product is filtered off with suction and dried in a drying oven until the weight remains constant. This gives the title compound (0.60 g) as a white crystalline solid.

MS: calc.: C₁₇H₁₈N₄O₄O₂ (380.13), found: [MH⁺]381.3

### 11. 2-(4-Difluoromethoxy-3-methoxybenzyl)-9-(2-oxypropyl)-1,9-dihydropurin-6-one

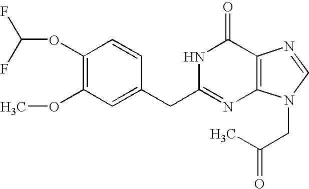

0.20 g (0.5 mmol) of 2-(4-difluoromethoxy-3-methoxybenzyl)-9-(2-hydroxypropyl)-1,9-dihydropurin-6-one is dissolved in 7 ml of absolute dichloromethane, 0.68 ml (4.8 mmol) of triethylamine is added and the mixture is cooled to 0° C. using an ice bath 2.3 ml of DMSO and 0.35 g (2.2 mmol) of pyridine/sulphur trioxide complex are added, and the mixture is then, under an atmosphere of nitrogen, stirred with ice-cooling for 1 h and at RT for a further 18 h. 20 ml of water are then added to the solution, and the mixture is extracted three times with dichloromethane. During the extraction, the product precipitates from the aqueous phase. The product is filtered off with suction and dried in a drying oven until the weight remains constant. This gives the title compound (0.12 g) as a white crystalline solid.

MS: calc.: C₁₇H₁₆N₄O₄F₂ (378.11), found: [MH⁺]379.3

### 12. 2-(3-Difluoromethoxy-4-methoxybenzyl)-9-(2-hydroxypropyl)-1,9-dihydropurin-6-one

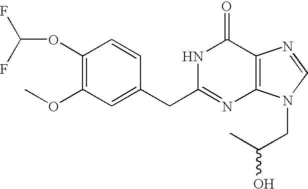

At RT, 2.3 g (8.8 mmol) of ethyl(3-difluoromethoxy-4-methoxyphenyl)acetate and 0.46 g (2.5 mmol) of 5-amino-1-(2-hydroxypropyl)-1H-imidazole-4-carboxamide 5 ml of warm ethanol are added to a solution of 0.25 g (11 mmol) of sodium in 15 ml of absolute ethanol, and the mixture is heated under reflux for 18 h. The ethanol is distilled off using a rotary evaporator, 20 ml of water are added to the residue and the mixture is then neutralized using 2N acetic acid. Precipitated product is filtered off with suction, recrystallized from ethyl acetate and dried in a drying oven at 40° C. until the weight remains constant. This gives the title compound (0.54 g) as a white crystalline solid.

MS: calc.: C₁₇H₁₆N₄O₄F₂ (380.13), found: [MH⁺]381.2

### 13. 2-(4-Difluoromethoxy-3-methoxybenzyl)-9-(2-oxypropyl)-1,9-dihydropurin-6-one

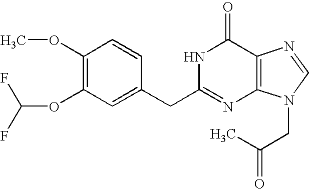

0.66 ml (4.75 mmol) of triethylamine and 7 ml of dichloromethane are added to 0.20 g (0.5 mmol) of 2-(4-difluoromethoxy-3-methoxybenzyl)-9-(2-hydroxypropyl)-1,9-dihydropurin-6-one, and the mixture is cooled to 0° C. using an ice bath. 2.33 ml of DMSO and 0.35 g ((2.2 mmol) of pyridine/sulphur trioxide complex are added and the mixture is then, under an atmosphere of nitrogen, stirred with ice-cooling for 1 h and at RT for a further 15 h. 20 ml of water are added to the orange solution, which is now turbid. During extraction with dichloromethane, the product precipitates out. Filtration with suction and drying in a drying oven gives the title compound (0.15 g) as a white crystalline solid.

MS: calc.: C₁₇H₁₆N₄O₄F₂ (378.11), found: [MH⁺]379.2

### 14. 2-(4-Difluoromethoxy-3-methoxybenzyl)-9-(2-hydroxypropyl)-8-phenyl-1,9-dihydropurin-6-one

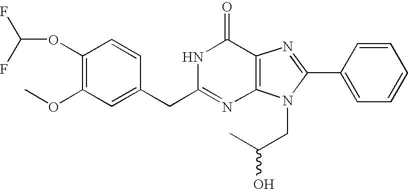

At RT, 3.7 g (14.4 mmol) of ethyl(4-difluoromethoxy-3-methoxyphenyl)acetate and 0.94 g (3.6 mmol) of 5-amino-1-(2-hydroxypropyl)-2-phenyl-1H-imidazole-4-carboxamide in 5 ml of warm ethanol are added to a solution of 0.42 g (18 mmol) of sodium in 35 ml of absolute ethanol, and the mixture is heated under reflux for 18 h. The ethanol is distilled off using a rotary evaporator, 20 ml of water are added to the residue and the mixture is neutralized using 2N acetic acid. Precipitated product is filtered off with suction and dried in a drying oven until the weight remains constant. This gives the title compound (0.560 g) as a white crystalline solid.

MS: calc.: C₂₃H₂₂N₄O₄F₂ (456.16), found: [MH⁺]457.3

### 15. 2-(4-Difluoromethoxy-3-methoxybenzyl)-9-(2-oxypropyl)-8-phenyl-1,9-dihydropurin-6-one

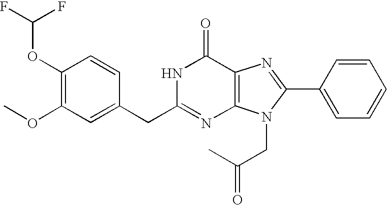

7 ml of absolute dichloromethane and 0.73 ml (5.2 mmol) of triethylamine are added to 0.25 g (0.55 mmol) of 2-(4-difluoromethoxy-3-methoxybenzyl)-9-(2-hydroxypropyl)-8-phenyl-1,9-dihydropurin-6-one, and the mixture is cooled to 0° C. using an ice-bath. 2.2 ml of DMSO and 0.35 g (2.2 mmol) of pyridine/sulphur trioxide complex are added, and the mixture is then, under an atmosphere of nitrogen, stirred with ice-cooling for 1H and at RT for another 15 h 20 ml of water are then added, and the mixture is extracted 3 times with in each case 25 ml of dichloromethane. The organic phases are washed with water and then dried over magnesium sulphate and concentrated using a rotary evaporator. The residue is triturated with ethyl acetate and dried in a drying oven, giving the title compound (0.20 g) as a white crystalline solid.

MS: calc.: C₂₃H₂₀N₄O₄F₂ (454.15), found: [MH⁺]455.3

### 16. 2-(4-Difluoromethoxy-3-methoxybenzyl)-9-(2-hydroxypropyl)-8-phenethyl-1,9-dihydropurin-6-one

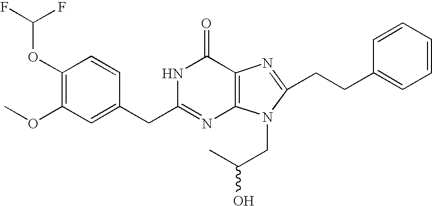

A solution of ethyl(4-difluoromethoxy-3-methoxyphenyl)acetate (2.34 g, 9 mmol) and 5-amino-1-(2-hydroxypropyl)-2-phenylethyl-1H-imidazole-5-carboxamide (865 mg, 3 mmol) in warm ethanol (20 ml) is added to a solution of sodium (345 mg, 15 mmol) in ethanol (20 ml), and the mixture is heated under reflux for 18 h. The ethanol is distilled off using a rotary evaporator, the residue is dissolved in dichloromethane (50 ml) and washed with water (20 ml) and most of the dichloromethane is removed using a rotary evaporator. The amorphous residue is recrystallized from 30% strength alcohol (60 ml). Drying gives the title compound as colourless crystals.

MS: calc.: C₂₅H₂₆F₂N₄O₄ (484.19), found: [MH⁺]485.3

### 17. 2-(4-Difluoromethoxy-3-methoxybenzyl)-9-(2-oxypropyl)-8-phenethyl-1,9-dihydropurin-6-one

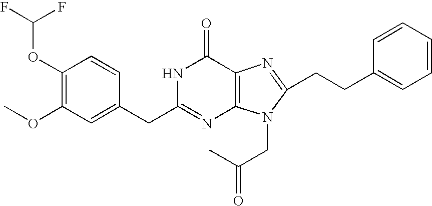

At <5° C., pyridine/sulphur trioxide complex 700 mg, 4.4 mmol) is added to a solution of 2-(4-difluoromethoxy-3-methoxybenzyl)-9-(2-hydroxypropyl)-8-phenylethyl-1,9-dihydropurin-6-one (484 mg, 1 mmol) in dichloromethane (20 ml), triethylamine (1.39 ml, 10 mmol) and DMSO (3 ml), and the mixture is stirred at <5° C. for 1 h and then at RT for 15 h. Most of the dichloromethane is removed from the reaction mixture, and the residue is triturated with water (20 ml), filtered off with suction and washed twice with water (in each case 10 ml). The amorphous powder is recrystallized from 70 ml of methanol. Filtration with suction and drying under a high vacuum gives the title compound (325 mg) as a colourless powder.

MS: calc.: C₂₅H₂₄F₂N₄O₄ (482.18), found: [MH⁺]483.4

### 18. 2-(4-Difluoromethoxy-3-methoxybenzyl)-9-(2-hydroxypropyl)-8-(3-phenylpropyl)-1,9-dihydropurin-6-one

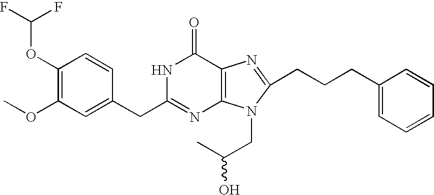

A solution of ethyl(4-difluoromethoxy-3-methoxyphenyl)acetate (1.30 g, 5.1 mmol) and 5-amino-1-(2-hydroxypropyl)-3-phenylpropyl-1H-imidazole-5-carboxamide (865 mg, 3 mmol) in warm ethanol (20 ml) is added to a solution of sodium (345 mg, 15 mmol) in ethanol (20 ml), and the mixture is heated under reflux for 16 h. The ethanol is distilled off using a rotary evaporator, the residue is dissolved in dichloromethane (50 ml) and washed with water (20 ml). Most of the dichloromethane is removed using a rotary evaporator, which gives the slightly contaminated title compound as light brown resin. After trituration with hot water (30 ml) and drying in an oven the title compound (452 mg) is received as off white powder.

MS: calc.: C₂₆H₂₈F₂N₄O₄ (498.21), found: [MH⁺]499.4

### 19. 2-(4-Difluoromethoxy-3-methoxybenzyl)-9-(2-oxypropyl)-8-phenylpropyl)-(3-phenylpropyl)-1,9-dihydropurin-6-one

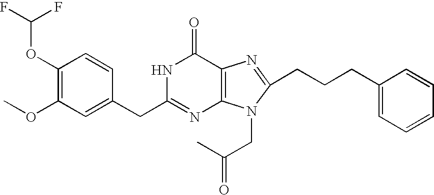

At <5° C., pyrridine/sulphur trioxide complex (350 mg, 2.2 mmol) is added to a solution of 2-(4-difluoro-3-methoxybenzyl)-9-(2-hydroxypropyl)-8-phenylpropyl-1,9-dihydropurin-6-one (249 mg, 0.5 mmol) in dichloromethane (10 ml), triethylamine (0.69 ml, 5 mmol) and DMSO (1.5 ml), and the mixture is stirred at <5° C. for 1 h and then at RT for 96 h. Most of the dichloromethane is removed from the reaction mixture, and the residue is triturated with hot water (10 ml), filtered off with suction and washed 4 times with water (10 ml each). The amorphous, colorless powder is dried under a high vacuum, which gives the title compound (208 mg).

MS: calc.: C₂₆H₂₅F₂N₄O₄ (498.19), found: [MH⁺]497.4

### 20. 2-(4-Difluoromethoxy-3-nitrobenzyl)-9-[1-(1-hydroxyethyl)-4-phenylbutyl]-1,9-dihydropurin-6-one

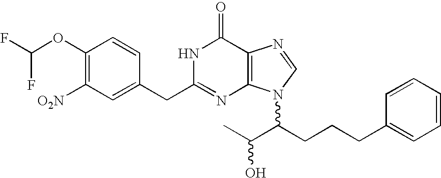

At RT, 9.6 g (34.8 mmol) of ethyl(4-difluoromethoxy-3-nitrophenyl)acetate and 3.9 g (12.9 mmol) of 5-amino-1-[1-(1-hydroxyethyl)-4-phenylbutyl]-1H-imidazole-4-carboxamide are added to a solution of 1.5 g (64.5 mmol) of sodium in 140 ml of ethanol, and the mixture is heated under reflux for 22 h. The ethanol is distilled off using a rotary evaporator, 30 ml of water are added to the residue and the mixture is neutralized with 2N acetic acid. The mixture is then extracted 3 times with in each case 200 ml of ethyl acetate. The extracts are washed with saturated sodium bicarbonate solution, the combined organic phases are dried over magnesium sulphate and the ethyl acetate is then removed using a rotary evaporator. Silica gel chromatography (ethyl acetate/methanol/NH₄OH=10:1:0.5) gives the title compound as a slightly contaminated, brown foam which, after trituration with isopropanol, is obtained as a virtually white, crystalline solid (0.8 g, 10%). Melting point: 195-198° C.

MS: calc.: C₂₅H₂₅F₂N₅O₅ (513.18), found: [MH⁺]514.3

### 21. 9-(1-acetyl-4-phenylbutyl)-2-(4-difluoromethoxy-3-nitrobenzyl)-1,9-dihydropurin-6-one

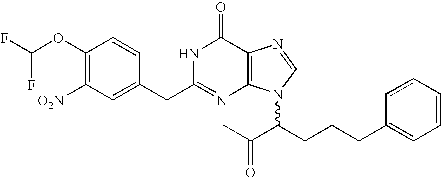

36 ml of absolute dichloromethane and 5.2 ml (37.0 mmol) of triethylamine are added to 2.0 g (3.9 mmol) of 2-(4-difluoromethoxy-3-nitrobenzyl)-9-[1-(1-hydroxyethyl)-4-phenylbutyl]-1,9-dihydropurin-6-one. With ice-cooling, 13 ml (151.1 mmol) of DMSO and 2.6 g (16.3 mmol) of pyridine/sulphur trioxide complex are added. Under an atmosphere of nitrogen, the mixture is then stirred at 0° C. for 1 h and at RT for a further 15 h. 20 ml of water are added to the solution, and the solution is made alkaline using 2N NaOH and is extracted twice with in each case 100 ml of dichloromethane. The organic phases are washed with water and then dried over magnesium sulphate and concentrated. Silica gel chromatography (ethyl acetate/methanol/NH₄OH=10:1:0.5) gives the title compound (1.25 g, 63%) as a brown solid.

MS: calc.: C₂₅H₂₃F₂N₅O₅ (511.17), found: [MH⁺]512.4

### 22. 9-(1-Acetyl-4-phenylbutyl)-2-(3-amino-4-difluoromethoxybenzyl)-1,9-dihydropurin-6-one

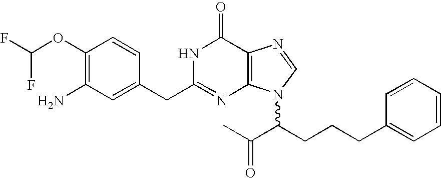

1.25 g (2.4 mmol) of 9-(1-acetyl-4-phenylbutyl)-2-(4-difluoromethoxy-3-nitrobenzyl)-1,9-dihydropurin-6-one are dissolved in 20 ml of methanol, 10 ml of glacial acetic acid, 4 ml of water and 120 mg of palladium on activated carbon (10%) are added and the mixture is hydrogenated at RT for 18 h. The mixture is filtered off with suction through Celite and the methanol is then evaporated. The residue is subsequently taken up in ethyl acetate and washed with saturated sodium bicarbonate solution, dried over magnesium sulphate and freed from the solvent Silica gel chromatography (ethyl acetate/methanol/NH₄OH=10:1:0.5) gives the title compound (0.27 g, 23%) as a beige crystalilne solid. Melting point: 149-150° C.

MS: calc.: C₂₅H₂₅F₂N₅O₃ (481.19), found: [MH⁺]482.3

### Synthesis of the Compounds 23-27

A solution of in each case 0.08 g (0.12 mmol) of 9-(1-acetyl-4-phenylbutyl)-2-(3-amino-4-difluoromethoxybenzyl)-1,9-dihydropurin-6-one (22) in a mixture of 2 ml of toluene and 1 ml of pyridine is admixed with 0.24 mmol of the acid chloride in question and stirred at 70° C. for 3 h. The solvent is removed using a rotary evaporator, in each case 10 ml of water are added to the residue and the mixture is acidified using 2N hydrochloric acid and extracted with 3×10 ml of dichloromethane. The combined organic phases are washed with saturated sodium bicarbonate solution and dried over magnesium sulphate, and the solvent is removed. The residue is then filtered through silica gel. In some cases, purification was carried out by preparative HPLC [gradient: t₀ acetonitrile/water=45:65, t_{1,5} acetonitrile/water 50:50, t_{5,5} acetonitrile/water=65:35]. After 12 h of freeze-drying, in each case white lyophilisates are obtained (Table 1).

### 23. N-[5-[9-(1-Acetyl-4-phenylbutyl)-6-oxo-6,9-dihydro-1H-purin-2-ylmethyl]-2-difluoromethoxybenzyl]-2-(4-methoxyphenyl)acetamide

### 24. N-[5-[9-(1-Acetyl-4-phenylbutyl)-6-oxo-6,9-dihydro-1H-purin-2-ylmethyl]-2-difluoromethoxybenzyl]-(4-methoxycarbonyl)benzamide

### 25. N-[5-[9-(1-Acetyl-4-phenylbutyl)-6-oxo-6,9-dihydro-1H-purin-2-ylmethyl]-2-difluoromethoxybenzyl]-(4-dipropylsulphamoyl)benzamide

### 26. N-[5-[9-(1-Acetyl-4-phenylbutyl)-6-oxo-6,9-dihydro-1H-purin-2-ylmethyl]-2-difluoromethyoxyphenyl]-3-chloro-4-fluorobenzamide

### 27. N-[5-[9-(1-Acetyl-4-phenylbutyl)-6-oxo-6,9-dihydro-1H-purin-2-ylmethyl]-2-difluoromethyoxyphenyl]-3-fluoro-4-methylbenzamide

TABLE 1

| | | | Summation | MS Fnd: |
|---|---|---|---|---|
| Example | Produkt | calc. | Formula | MH⁺ |
| 23 | 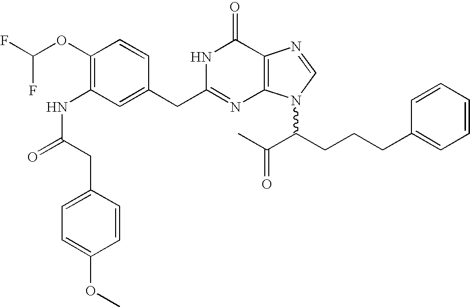 | 629,67 | C₃₄H₃₃F₂N₅O₅ | 630,4 |
| 24 | 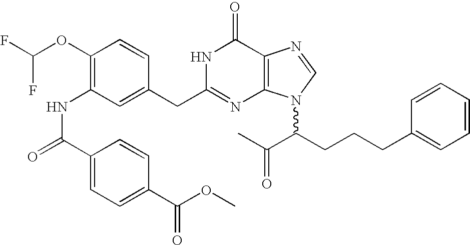 | 643,65 | C₃₄H₃₁F₂N₅O₆ | 644,3 |
| 25 | 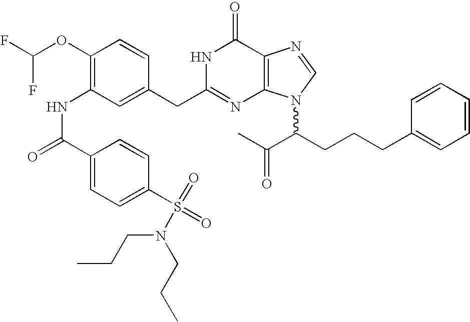 | 748,86 | C₃₃H₄₂F₂N₅O₆S | 749,4 |
| 26 | 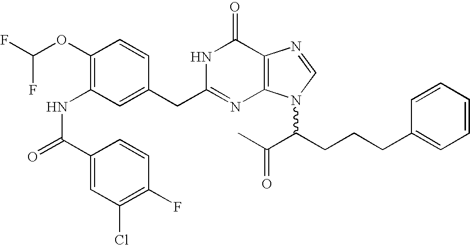 | 638,05 | C₃₂H₂₇ClF₃N₅O₄ | 638,3 |
| 27 | 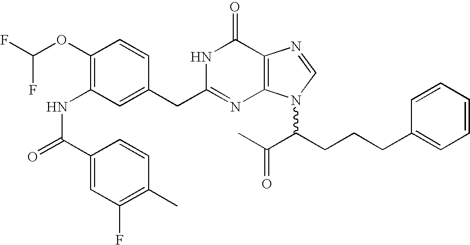 | 617,63 | C₃₃H₃₀F₃N₅O₄ | 618,3 |

### 28. 2-[4-Chloro-3-(difluoromethoxy)-benzyl]-9-[1-(1-hydroxy-ethyl)-4-phenyl-butyl]-1,9-dihydropurin-6-one

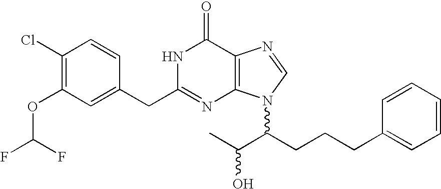

A solution of ethyl(3-difluoromethoxy-4-chlorphenyl)acetate (1.32 g, 5 mmol) and 5-amino-1-[1-(1-hydroxyethyl)-4-phenylbutyl]-1H-imidazole-4-carboxamide (605 mg, 2 mmol) in warm ethanol (10 ml) is added to a solution of sodium (345 mg, 15 mmol) in ethanol (10 ml), and the mixture is heated under reflux for 24 h. The ethanol is distilled off using a rotary evaporator, the residue is dissolved in saturated bicarbonate solution (30 ml), water (30 ml) and ethylacetate (30 ml). The phases are separated and the water layer is extracted twice with ethylacetate (30 ml each). The combined organic layers are dried over magnesium sulphate and the solvent is removed off. Silica gel chromatography (90 g of silica gel, ethyl acetate/methanol =10/1), crystallization from diethylether and drying under reduced pressure gives the title compound (587 ° mg) as colorless crystals.

MS: calc.: C₂₅H₂₃F₂N₄O₃ (502.18), found: [MH⁺]503.2

### 29. 9-(1-Acetyl-4phenyl-butyl)-2-[4-chloro-3-(difluoromethoxy)-benzyl]-1,9-dihydro-purin-6-one

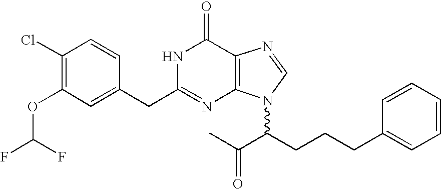

At <5° C. pyridine/sulphur trioxide complex (350 mg, 2.2 mmol) is added to a solution of 2-[4-Chloro-3-(difluoromethoxy)-benzyl]-9-[1-(1-hydroxy-ethyl)-4-phenyl-butyl]-1,9-dihydropurin-6-one (251 mg, 0.5 mmol) in dichloromethane (10 ml), triethylamine (1.39 ml, 10 mmol) and DMSO (2 ml), and the mixture is stirred at <5° C. for 1 h and then at RT for 15 h. The reaction mixture is quenched with water (20 ml), the phases are separated and the aqueous layer extracted twice with dichloromethane (20 ml each). The combined organic layers are dried over magnesium sulphate. After evaporation of the solvent, silica gel chromatography (50 g of silica gel, ethylacetate/methanole=10/1) and drying under high vacuum gives the title compound (180 mg) as a colorless resin.

MS: calc.: C₂₅H₂₃F₂N₄O₃ (500.14), found: [MH⁺]501.3

Ausgangsverbindungen:

### A1. 5-Amino-1-[1-(1-hydroxyethyl)-4-phenylbutyl]-1H-imidazole-4-carboxamide

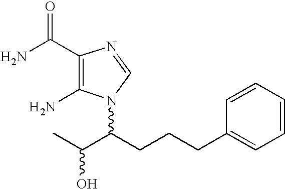

2-amino-2-cyanoacetamide (2.65 g, 26.7 mmol) is coevaporated twice with toluene (in each case 20 ml) and then suspended in absolute acetonitrile. Triethyl orthoformate (5.0 ml, 30 mmol) is added and the reaction mixture is heated for 1 h under reflux. 3-Amino-6-phenylhexan-2-ol (7.7 g 40 mmol) in acetonitrile (20 ml) is added and heating under reflux is continued for further 15 min. After cooling, the acetonitrile is distilled off using a rotary evaporator. Silica gel chromatography (200 g, ethyl acetate/methanol=10:1), recrystallisation from ethylacetate (60 ml) and drying under reduced pressure gives the title compound as colorless crystals (4.95 g). Melting point: 132° C.

MS: calc.: C₁₅H₂₂N₄O₂ (302.17), found: [MH⁺]303.2

### A2. 5-Amino-1-[1-(1-hydroxyethyl)-4-phenylbutyl]-2-methyl-1H-imidazole-4-carboxamide

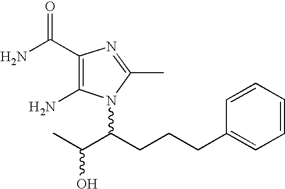

A suspension of 1.49 g of 2-amino-2-cyanoacetamide (15.0 mmol) in 50 ml of absolute acetonitrile is heated with 2.92 ml of triethyl orthoacetate (16.0 mmol) for 1 h under reflux. 2.9 g of 3-amino-6-phenylhexan-2-ol (15.0 mmol) in 15 ml of absolute acetonitrile are added, and the mixture is then heated under reflux for a further 15 min. After cooling, the acetonitrile is distilled off using a rotary evaporator. Silica gel chromatography (200 g, ethyl acetate/methanol=10:1) gives the title compound (1.27 g) as a light-yellow solid.

MS: calc.: C₁₇H₂₄N₄O₂ (316.19), found: [MH⁺]317.1

### A3. 3-Amino-6-phenylhexan-2-ol

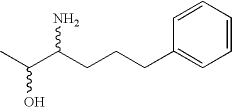

A solution of 3-phenylpropylmagnesiumbromide is prepared from 3-phenylpropane (35.6 g, 179 mmol) and magnesium (4.7 g, 195 mmol) in ether (100 ml). To this solution 2-trimethylsilyloxypropionitril (23.3 g, 162 mmol) in ether (100 ml) is added dropwise and the mixture is refluxed for 1 h. After cooling to RT a solution of sodiumboranate (6.8 g, 179 mmol) in ethanol (200 ml) is added dropwise and the mixture is then refluxed for 3 h and afterwards stirred for 16 h at RT. Water (50 ml) is added dropwise, followed by 4molar hydrochloric acid (50 ml). Phases are separated and the organic layer is extracted with 2-molar hydrochloric acid (100 ml). After treatment with potassiumhydroxyde solution (20%) and solid potassiumoarbonate, the mixture is extracted 4 times with chloroform in each case 100 ml), dried over potassium carbonate, filtered and the solvent is removed in vacuum to give the crude product (13.8 g), which is used without purification for the next step.

MS: calc.: C₁₂H₁₉NO (193.15), found: [MH⁺]194.2

### 4. 5Amino-1-[2-hydroxypropyl)-1H-imidazole-4-carboxamide

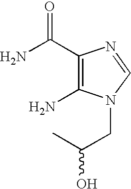

A suspension of 2.48 g of 2-amino-2-cyanoacetamide (25.0 mmol) in 90 ml of absolute acetonitrile is heated with 3.71 g of triethyl orthoformate (25.0 mmol) for 1 h under reflux. 1.88 g of 1-amino-2-propanol (25.0 mmol) in 20 ml of absolute acetonitrile are added, and the mixture is then heated under reflux for a further 15 min. After cooling, the acetonitrile is distilled off using a rotary evaporator. Silica gel chromatography (150 g, ethyl acetate/methanol=10:1) gives the title compound (2.5 g) as a white crystalline solid.

MS: calc.: C₇H₁₂N₄O₂ (184.10), found: [MH⁺]185.1

### A5. 5-Amino-1-(2-hydroxypropyl)-2-phenyl-1H-imidazole-4-carboxamide

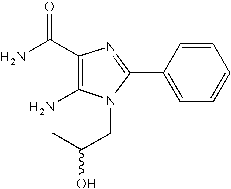

A suspension of 2.48 g of 2-amino-2-cyanoacetamide (25.0 mmol) in 90 ml of absolute acetonitrile is heated with 5.6 g of triethyl orthobenzoate (25.0 mmol) for 1 h under reflux. 1.88 g of 1-amino-2-propanol (25.0 mmol) in 20 ml of absolute acetonitrile are added, and the mixture is then heated under reflux for a further 15 min. After cooling, the acetonitrile is distilled off using a rotary evaporator. Silica gel chromatography (220 g, ethyl acetate/methanol=10:1) gives the title compound (1.42 g) as a dark red/brown foam.

MS: calc.: C₁₃H₁₆N₄O₂ (260.13), found: [MH⁺]261.1

### A6. 5-Amino-1-[1-(2-hydroxypropyl)-2-phenethyl]-1H-imidazole-4-carboxamide

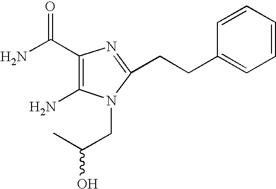

A suspension of 2-amino-2-cyanoacetamide (2.97 g, 30 mmol) in acetonitrile (60 ml) is heated with (3,3,3-triethoxy-propyl)-benzene (7.6 g, 30 mmol) for 1 h under reflux. Aminopropan-2-ol (2.5 ml, 31.5 mmol) is then added, and the mixture is heated under reflux for a further 15 min. The acetonitrile is distilled off using a rotary evaporator. Silica gel chromatography (200 g of silica gel, ethyl acetate/methanol=10/1), recrystallisation from ethyl acetate (130 ml) and drying under reduced pressure gives the title compound (3.7 g) as colorless crystals.

MS: calc.: C₁₅H₂₀N₄O₂ (288.16), found: [MH⁺]289.1

### A7. 5-Amino-1-(2-hydroxypropyl)-2-(3-phenylpropyl)-1H-imidazole-4-carboxamide

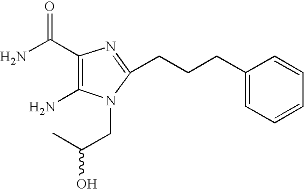

A suspension of 2-amino-2-cyanoacetamide (2.97 mmol, 30 mmol) in acetonitrile (100 ml) is heated with (4,4,4-triethoxy-butyl)-benzene (9.3 g, 35 mmol) for 1 h under reflux. Aminopropan-2-ol (2.5 ml, 31.5 mmol) is then added, and the mixture is heated under reflux for a further 15 min. The acetonitrile is distilled off using a rotary evaporator. Silica gel chromatography (200 g of silica gel, ethyl acetate/methanol=10/1), recrystallisation from ethylacetate (60 ml) and drying under reduced pressure gives the title compound (4.44 g) as colourless crystals.

MS: calc.: C₁₆H₂₂N₄O₂ (302.17), found: [MH⁺]303.1

### A8. Ethyl(3-difluoromethoxy-4-methoxyphenyl)acetate

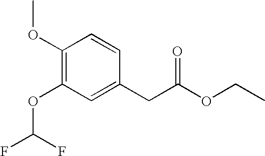

Concentrated sulphuric acid (1 ml) is added to a solution of (3-difluoromethoxy-4-methoxyphenyl)acetic acid (14.2 g, 51 mmol) in ethanol (140 ml), and the mixture is heated under reflux for 3 h. The solvent is removed using a rotary evaporator and the residue is dissolved in ethyl acetate (200 ml). The solution is washed with water (50 ml), saturated sodium bicarbonate solution (50 ml) and saturated sodium chloride solution (50 ml). The solution is dried over magnesium sulphate, the ethyl acetate is removed and the residue is then distilled under a high vacuum (b.p._{0.07} 115-119° C). This gives the title compound (15.0g) as a colourless oil.

MS: calc.: C₁₂H₁₄F₂O₄ (260.09), found: [MH⁺]260

### A9. Ethyl(4-difluoromethoxy-3-methoxyphenyl)acetate

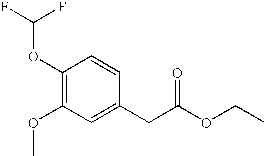

Concentrated sulphuric acid (1 ml) is added to a solution of (4-difluoromethoxy-3-methoxyphenyl)acetic acid (11.6 g, 50 mmol) in ethanol (50 ml), and the mixture is heated under reflux for 3 h. The solvent is removed using a rotary evaporator, and the residue is dissolved in ethyl acetate (200 ml). The solution is washed with water (50 ml), saturated sodium bicarbonate solution ((50 ml) and saturated sodium chloride solution (50 ml). The solution dried over magnesium sulphate, the ethyl acetate is removed and the residue is then distilled under a high vacuum (b.p._{0.05} 100-109° C.). This gives the title compound (9.7 g) as a colourless oil.

MS: calc.: C₁₂H₁₄F₂O₄ (260.09), found: [MH⁺]261

### A10. (3-Difluoromethoxy-4-methoxyphenyl)acetate acid

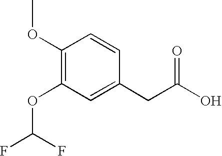

With vigorous stirring, gaseous chlorodifluoromethane is added at 65° C. to a solution of homoisovanillic acid (17.5 g, 96 mmol) and potassium hydroxide (54 g, 960 mmol) in water (108 ml) and dioxane (500 ml). After. 1 h, potassium hydroxide (393 g, 7 mol) in water (590 ml) is added dropwise over a period of 5 h to the solution, which continues to be treated with gas. Stirring is continued for 1 h, and the mixture is then cooled. The solution is acidified with citric acid (about 500 g) and extracted three times with ethyl acetate (in each case 300 ml). The combined organic phases are washed twice with water (in each case 200 ml), and with saturated sodium chloride solution (200 ml). The mixture is re-extracted twice with dilute ammonia solution (in each case 250 ml; about 10%), and the ammonia phases are evaporated to dryness using a rotary evaporator. The residue is dissolved in water (150 ml), adjusted to a pH of about 9 using ammonia solution, filtered and acidified using citric acid (about 30 g), and the carboxylic acid crystallizes out following seeding. Drying gives the title compound (9.3 g) as colourless crystals.

MS: calc.: C₁₀H₁₀F₂O₄ (232.05), found: [MH⁺]232

### A11. (4-Difluoromethoxy-3-methoxyphenyl)acetate acid

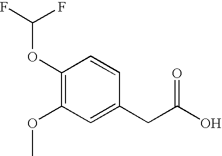

With vigorous stirring, gaseous chlorodifluoromethane is introduced at 65° C. into a solution of homovanillic acid (0.91 g, 5 mmol) and potassium hydroxide (2.8 g, 50 mmol) in water (5.6 ml) and dioxane (10 ml). After 1 h, potassium hydroxide (28 g, 500 mmol) in water (56 ml) is added dropwise over a period of 5 h to the solution, which continues to be treated with gas. The mixture is stirred for another hour and then cooled. The solution is acidified with citric acid (about 25 g) and extracted three times with ethyl acetate (in each case 20 ml). The combined organic phases are washed twice with water (in each case 20 ml), and with saturated sodium chloride solution (20 ml), and dried over magnesium sulphate, and the solvent is removed using a rotary evaporator. The amorphous residue is recrystallized from toluene (15 ml), resulting in the recovery of homovanillic acid (0.28 g). The mother liquor is concentrated using a rotary evaporator and the amorphous residue is recrystallized from water (30 ml), filtered off with suction and washed with water (10 ml). Drying gives the We compound (0.384 g) as colourless crystals.

MS: calc.: C₁₀H₁₀F₂O₄ (232.05), found: [MH⁺]232

### A12. Ethyl(4-difluoromethoxy-3-nitrophenyl)acetate

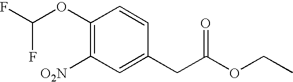

1.6 ml of concentrated sulphuric acid are added to a solution of 6.0 g (24.3 mmol) of (3-difluoromethoxy-4-methoxyphenyl)acetic acid in 4 ml of ethanol, and the mixture is heated under reflux for 3.5 h. The solvent is removed using a rotary evaporator and the residue is taken up in 150 ml of ethyl acetate. The mixture is washed with 50 ml of saturated sodium bicarbonate solution. The organic phase is dried over magnesium sulphate and the ethyl acetate is removed, giving the title compound (6.3 g, 94%) as a dark-brown oil.

MS: calc.: C₁₁H₁₁F₂NO₅ (275.06), found: [MH⁺]275.1

### A13. (4-Difluoromethoxy-3-nitrophenyl)acetate acid

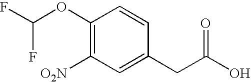

19.1 g (100 mmol) of (4-hydroxy-3-nitrophenyl)acetic acid and 28 g (500 mmol) of potassium hydroxide are dissolved in 60 ml of water and 800 ml of dioxane. With stirring at 50° C., Frigen 22 (CHClF₂) is introduced for 1 h. Subsequently, over the course of 8 h, 560 g (10.0 mol) of potassium hydroxide in 1.2 l of water are added dropwise, also at 50° C., and the mixture is stirred for another 6 h with introduction of gas. After cooling, the phases are separated. The organic phase is concentrated, the residue is dissolved in a solution of 2.0 g (36.0 mmol) of potassium hydroxide in 150 ml of water, activated carbon is added and the solution is filtered off. The solution is then adjusted to pH 2.5 using citric acid (99% strength) and heated at 60° C. The substance that precipitates is filtered off with suction and washed with water. The aqueous phase is acidified with citric acid (99% strength) and extracted 3 times with in each case 300 ml of ethyl acetate. Combined organic phases are washed with water, dried over magnesium sulphate and concentrated. The product-containing fractions obtained after column chromatography (ethyl acetate/methanol/NH₄OH=6:3:1) are freed from the solvent, the residue is dissolved in water and acidified with citric acid (99% strength) and precipitated product is filtered off with suction. Drying of the two fractions gives the title compound (14.9 g, 60%) as a lightbrown solid.

MS: calc.: C₉H₇F₂NO₈ (247.16), found: [MH⁺]245.9

### A14. Ethyl-(4-chloro-3-difluoromethoxyphenyl)acetate

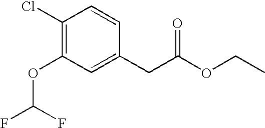

Concentrated sulphuric acid (1 ml) is added to a solution of (4-chloro-3-difluoromethoxyphenyl)acetate acid (13.84 g, 58.5 mmol) in ethanol (70 ml), and the mixture is heated under reflux for 4 h. The solvent is removed using a rotary evaporator, and the residue is dissolved 1 h ethyl acetate (100 ml). The solution is washed with water (50 ml, saturated sodium bicarbonate solution (50 ml and saturated sodium chloride solution (50 ml). The solution is dried over magnesium sulphate and the ethyl acetate is removed, giving the title compound (14.2 g) as a colorless oil.

MS: calc.: C₁₁H₁₁ClF₂O₃ (264.04), found: [MH⁺]264.1

### A15. (4-Chloro-3-difluoromethoxyphenyl)acetate acid

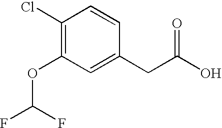

With vigorous stirring, Frigen 22 (CHClF₂) is introduced at 50° C. into a solution of (3-chloro-4-hydroxyphenyl)acetate acid (18.7 g, 100 mmol) and potassium hydroxide (28 g, 500 mmol) in water (56 ml) and dioxane (800 ml) over a period of 1 h. Subsequently, potassium hydroxide (560 g, 10 mol) in water (1.12 l) is added dropwise over a period of 7 h, and the mixture is stirred for another 6 h with introduction of gas. After 1 additional hour of stirring, the mixture is cooled and the phases are separated. The organic phase is concentrated, the residue is dissolved in water (150 ml) cleared with Tonsil and acidified with acetic acid. The precipitated substance is filtered with suction and subsequent drying gives the title compound (7.9 g) as colorless crystals. The aqueous phase is acidified with citric acid (ca 500 g) and extracted twice with ethylacetate (400 ml). The combined organic layers are washed with water and evaporated. The residue is recrystallized twice from water (150 ml each). Drying gives a second crop of the title compound (6.0 g) as colorless crystals.

MS: calc.: C₉H₇ClF₂O₃ (236.00), found: [MH⁺]236

Commercial Applicability

The compounds according to the invention have useful pharmacological properties which make them industrially utilizable. As selective cyclic nucleotide phosphodiesterase (PDE) inhibitors (specifically of type 2), they are suitable on the one hand as therapeutics for conditions of pathologically enhanced endothelial activity and impaired endothelial barrier function such as septic shock, vascular edema, or diseases associated with unwanted neoangiogenesis. On the other hand, given the expression of PDE2 in neuronal tissue the compounds may also be useful in neurodegenerative conditions. In addition, PDE2 is expressed in human platelets and PDE2 inhibitors were shown to suppress platelet functions. In consequence, the compounds may be used as anti-thrombotics/platelet aggregation inhibitors. Furthermore, since PDE2 was shown in myocardium the compounds may afford a potential to protect against arrhythmias.

On account of their PDE2-inhibiting properties, the compounds according to the invention can be employed in human and veterinary medicine as therapeutics, where they can be used, for example, for the treatment and prophylaxis of the following illnesses: (1) all conditions of pathologically enhanced endothelial activity/impaired endothelial barrier function such as multi-organ failure in particular acute respiratory distress syndrome (ARDS) in septic shock, pneumonia, acute and chronic airway disorders of varying origin (rhinitis, bronchitis, bronchial asthma, emphysema, COPD), angioedema, peripheral edema, cerebral edema for example traumatic or following stroke; (2) all conditions associated with pathologically enhanced neoangiogenesis such as all kinds of tumors (benign or malignant) which are associated with neoangiogenesis and all kinds of inflammatory diseases associated with neoangiogenesis for example disorders of the arthritis type (rheumatoid arthritis, rheumatoid spondylitis, osteoarthritis and other arthritic conditions), all forms of psoriasis, retinal blindness, bronchial asthma, inflammatory bowel disease, transplant rejection, allograft rejections, atherosclerosis; (3) all conditions for which platelet aggregation inhibition in conjunction with reduction of enhanced endothelial actuation is desireable such as thrombembolic disorders and ischaemias covering myocardial infarct cerebral infarct, transitory and ischaemic attacks, angina pectoris, peripheral circulatory disorders, prevention of restenosis after thrombolysis therapy, percutaneous translumial angioplasty (PTA), percutaneous transluminal coronary angioplasty (PTCA) and bypass; (4) all types of impaired cognition in particular cognitive disorders such as mild cognitive disorder (MCI), Alzheimer's disease, Lewy-Body dementia, Parkinson's disease and cerebrovascular dementia; and (5) in cardiac arrhythmias.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 2 (PDE2), ameliorating the symptoms of an PDE2mediated disorder, and wherein the packaging material comprises a label or package insert which indicates that the pharmaceutical agent is useful for preventing or treating PDE2-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Intravenous and oral delivery is preferred.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel farmers, ointment bases and other active compound excipients, for example antioxidants, dispersants, emulsifiers, preservatives, solubilizers, colorants, complexing agents or permeation promoters, can be used.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP D 505 321), using which an optimal administration of active compound can be achieved.

For the treatment of skin diseases, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarly between 0.1 and 3 mg per day. The customary dose in the case of systemic therapy (p.o. or i.v.) is between 0.03 and 3 mg/kg per day.

Biological Investigations

Method for Measuring Inhibition of PDEs Activities

Abbreviations:

PDE: phosphodiesterase, PCR: polymerase chain reaction, RT-PCR: reverse transcription-polymerase chain reaction, dNTPs: deoxynucleoside triphosphates, RNA: ribonucleic acid, cDNA: complementary deoxyribonucleic acid, bp: basepairs, (dT)₁₅: pentadecathymidylic acid, ORF: open reading frame, GB no.: GenBank database accession number, rBV: recombinant baculovirus, wt: wild type, aa: aminoacid, UCR: upstream conserved region, PAA: polyacrylamide.

Aminoacids are abbreviated with the 1-character symbol: A for alanine, C for cysteine, D for aspartic acid, E for glutamic acid, F for phenylalanine, G for glycine, H for histidine, I for isoleucine, K for lysine, L for leucine, M for methionine, N for asparagine, P for proline, Q for glutamine, R for arginine, S for serine, T for threonine, V for valine, W for tryptophane, Y for tyrosine.

General Methods for Cloning Recombinant PDEs

RNA was purified from cell lines using the RNeasy Mini Kit from Qiagen. 1 μg RNA was reverse transcribed into single-stranded cDNA in a 20 μl reaction using Expand Reverse Transcriptase (Roche) with 50 pM of primer (dT)₁₅ and 1 mM dNTPs (both from Roche). 5 μl of cDNA were used as template for the subsequent PCR reaction. Human cDNAs from tissues were purchased from Clontech or Invitrogen. 1 μl was used for PCR reaction.

PCR was carried out in a Stratagene Robocycler 40 or in a MWG Primus 96 plus thermocycler. Typically, PCR was carried out with the Expand Lond Template PCR System from Roche in buffer 3 plus 0.75 mM MgCl₂, 0.3 μM each primer, 500 μM dNTPs.

PCR products were purified with the High Pure PCR Product Purification Kit (Roche) or from agarose gel with the QlAquick Gel Extraction kit from Qiagen, and cloned into the pCR2.1-TOPO vector from invitrogen. The ORFs were subcloned in baculovirus expression vectors (transfer plasmids). The pCR-Bac and pVL vectors were from invitrogen. The pBacPak vectors (pBP8 or pBP9) were from Clontech. Restriction endonucleases were from Roche and MBI Fermentas. Modifying enzymes and T4 DNA ligase were from New England Biolabs. DNA was sequenced by the company GATC GmbH (Konstanz, Germany, www.gate.de) or in ALTANA Pharma's lab using an ABI PRISM 310 and the Big dye terminator cycle sequencing v2 chemistry (Applied Biosystem). Sequence analysis was performed with Hitachi Software

DNASIS Version 2.5 or with Vector NTI 7. When necessary, in vitro mutagenesis was eventually performed with the QuickChange Site-Directed Mutagenesis Kit from Stratagene.

Cloning of human PDE 2A3

The PDE2A3 (GB no. U67733) was amplified in 2 steps using PCR from brain EDNA. A N-terminal fragment was isolated using primers CP1PD2AS (5′-GAGGAGTGATGGGGCAGGC-3′) and PR9PD2AA (5′-GCGAAGTGGGAGACAGAAAAG-3′), a C-terminal fragment was isolated using primers PR7PD2AS (5′- GATCCTGAACATCCCTGACG-3′) and CP3PD2AA (5′GGGATCACTCAGCATCAAGGC-3). The PCR products were cloned into the vector pCR2.1-Topo. The N-terminal fragment was first subcloned with EcoRI into pBluescript II KS (−), afterwards a Bst1 107l/EcoRV fragment was exchanged with the corresponding restriction fragment from the C-terminal clone, to obtain a complete ORF. The ORF for the PDE2A3 was subcloned into pBP8 using XbaI and KpnI.

Expression of Recombinant PDE2

The rBV was prepared by means of homologous recombination in Sf9 insect cells. The expression plasmids were cotransfected with Bac-N-Blue (Invitrogen) or Baculo-Gold DNA (Pharmingen) using a standard protocol (Pharmingen). Wt virus-free recombinant virus supernatants were selected using plaque assay methods. After that, high-titre virus supernatants were prepared by amplifying 3 times. PDE2 was expressed in Sf21 cells by infecting 2×10⁶ cells/ml with an MOI (multiplicity of infection) between 1 and 10 in serum-free SF900 medium (Life Technologies). Cells were cultured at 28° C., typically for 48 hours, after which they were pelleted for 5-10 min at 1000 g and 4° C. In spinner flasks, cells were cultured at a rotational speed of 75 rpm. The SF21 insect cells were resuspended, at a concentration of approx. 10⁷ cells/ml, in ice-cold (4° C.) homogenization buffer (20 mM Tris, pH 82, containing the following additions: 140 mM NaCl, 3.8 mM KCl, 1 mM EGTA, 1 mM MgCl₂, 1 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000×g and the supernatant was stored at −80° C. until subsequent use (see below). The protein content was determined by the Bradford method (BioRad, Munich) using BSA as standard. Integrity and size of recombinant proteins were analysed by western blot.

Measurement of Recombinant Human PDE2A3 Inhibition by SPA Technology

Recombinant human PDE2A3 activities were inhibited by the compounds according to the invention in a modified SPA (scintillation proximity assay) test, supplied by Amersham Pharmacia Biotech (see procedural instructions “phosphodiesterase[3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 5 μM cGMP (to activate PDE2A3), 2 μl of the respective substance dilution in DMSO and sufficient recombinant PDE (1000×g supernatant, see above) to ensure that 15-20% of the cAMP is co varied under the said experimental conditions. After a preincubation of 5 min at 37° C., the reaction is started by adding the substrate (cAMP) and the assays are incubated for a further i5 min; after that, they are stopped by adding SPA beads (50 μl). In accordance with the manufacturer's instructions, the SPA beads had previously been resuspended in water and then diluted 1:3 (v/v); the diluted solution also contains 3 mM IBMX. After the beads have been sedimented (>30 min), the MTP's are analyzed in commercially available measuring appliances and the corresponding IC₅₀ values of the compounds for the inhibition of PDE activities are determined from the concentration-effect curves by means of non-linear regression.

Method to Assess Inhibition of Macromolecule Permeability of HUVEC Monolayers:

The procedure to measure macromolecule permeability of endothelial cell monolayers followed the method described by Langeler & van Hinsbergh (1988) with modifications. Human umbilical vein endothelial cells were isolated from umbilical cords according to standard procedures (Jaffe et al. 1973) and cultured in endothelial cell basal medium (EBM) supplemented with 2% FCS, 0.5ng/ml VEGF, 10 ng/ml bFGF, 5 ng/ml EGF, 20 ng/ml Long R3 IGF-1, 0.2 μg/ml hydrocortisone, 1 μg/ml ascorbic acid, 22.5 μg/ml heparin, 50 μg/ml gentamicin, 50 ng/ml amphotericin B (EGM2 purchased from Promocell GmbH, Heidelberg, Germany). At confluency, cells were trypsinized and replated at 73000 cells per well on 3 μm polycarbonate filter Transwell inserts (Costar GmbH, Bodenheim, Germany) precoated with 10 μg cm²⁻¹ Fl-bronectin (Sigma, Taufkirchen, Germany). HUVECs were cultured in EGM (100 μl in the upper wells and 600 μl in the lower wells) over four days prior the experiments and medium was changed every other day. At the day of the experiment culture medium was replaced by M199 with 1% human serum albumin. Endothelial cells were preincubated with cyclic nucleotide modifiers (the selective PDE3 inhibitor motapizone, the selective PDE4 inhibitor RP73401, the cGMP generators ANP or SNP and PDE2 inhibitors) for 15 min. HUVECs were then stimulated with Thrombin (1 μm⁻¹) (Sigma, Taufkirchen, Germany) and horsh radish peroxidase (5 μg/ml) (Sigma, Taufkirchen, Germany) as the macromolecule marker protein was added to the upper wells. Following 1 h incubation time Transwells were removed and the activity of horsh radish peroxidase that penetrated the endothelial call monolayer was measured in the lower wells with the 3,3′, 5,5′-tetramethylbenzidine liquid substrate system from Sigma (Taufkirchen, Germany).

The compounds according to the invention potently suppress recombinant human PDE2A3 activity. The inhibitory values [measured as −log IC₅₀ (mol/l)] determined for the examples 1 to 9 and 23 to 29 are higher than 7.5

In parallel, the compounds according to the invention inhibited Thrombin-induced permeability of HUVEC monolayers for horsh radish peroxidase (HRP) as a macromolecule marker. Therefore, PDE2 inhibitors are suggested to improve the endothelial barrier function, which is impaired in numerous conditions such as acute respiratory distress syndrome (ARDS) or severe pneumonia. The system to measure these cellular effects of the PDE2 inhibitors observed the enzymological characteristics of PDE2 which exhibits a rather high Km for CAMP and the activity of which is activated by cGMP. The Thrombin-induced increase of HRP permeability was completely abolished by complete inhibition of PDE3 (10 μM Motapizone) and PDE4 (1 μM RP73401). However, in the additional presence of ANP (100 nM) or SNP (1 mM) to augment cGMP the inhibition by PDE3 and 4 inhibition of permeability was partially reversed. PDE2 inhibitors blocked the thrombin-stimulated HRP-permeability if 1 μM RP73401, 10 μM Motapizone, 100 nM ANP or 1 mM SNP were present indicating that ANP or SNP by generating cGMP activate PDE2. The concentration-dependent inhibition of HRP permeability at different concentrations of example 5 was assessed from the percent inhibition in the presence and absence of the PDE2 inhibitors and in the presence of 1 μM RP73401, 10 μM Motapizone and 100 nM ANP. In the absence of PDE3 and 4 inhibition, ANP or SNP the PDE2 inhibitors showed very little effect in Thrombin-Induced macromolecule hyperpermeability.

[image]

[image]

FIG. 1A/B: HUVEC cells on 3 μm polycarbonate filters (Transwells) were preincubated with 1 μM RP73401 (RP, to block PDE4) and 10 μM Motapizone (M, to block PDE3), 1 mM SNP or 100 nM ANP and 1 μM of example 5 over 15 min and then stimulated with 1U/ml thrombin. HRP passage into the lower wells was assessed after 60 min. RP73401 and Motapizone completely blocked thrombin-induced hyperpermeability, which was partially reversed by SNP and ANP. Example 5 inhibited this SNP- or ANP-induced permeability increase in a concentration-dependent fashion (FIG. 1B).

1. Compounds of formula 1 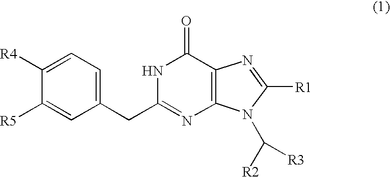
in which
R1 is hydrogen, 1-4C-alkyl, phenyl or phenyl-1-4C-alkyl,
and in which either
R2 is 1-4C-alkyl, 1-hydroxy-2-4C-alkyl, 1-4C-alkylcarbonyl or 1-(acetyloxy)-2-4C-alkyl and
R3 is hydrogen,
or
R2 is hydrogen, 1-4C-alkyl, 1-hydroxy-2-4C-alkyl, 1-4C-alkylcarbonyl or 1-(acetyloxy)-2-4C-alkyl and
R3 is Arylbutyl, Heteroarylbutyl, Arylpropyl, Heteroarylpropyl, Arylethyl or Heteroarylethyl,
wherein
Aryl is phenyl, naphthalenyl or indanyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl,
Heteroaryl is pyridinyl, pyrazinyl, pyridazinyl, pyrimidinyl, quinazolinyl, quinoxalinyl, cinnolinyl, quinolyl, isoquinolyl, naphthyridinyl, phthalazinyl, indolyl, isoindolyl, indazolyl, purinyl, pteridinyl, benzofuranyl, benzoxazolyl, benzothiazolyl, benzimidazolyl, oxazolyl, isoxazolyl, thiazolyl, isothiazolyl, imidazolyl, pyrrolyl, pyrazolyl, furanyl or thiophenyl, each of which optionally substituted up to three times identically or differently by halogen, hydroxyl, nitro, trifluoromethyl, carboxyl, 1-4C-alkyl, 1-4C-alkoxy or 1-4C-alkoxycarbonyl,
and in which either
R4 is 1-4C-alkoxy which is completely or predominantly substituted by fluorine and
R5 is halogen, hydroxyl, nitro, trifluoromethyl, carboxyl, 1-4C-alkyl, 1-4C-alkoxy, 1-4C-alkoxy which is completely or predominantly substituted by fluorine, 1-4C-alkoxycarbonyl, amino, mono- or di-1-4C-alkylamino, aminocarbonyl, mono- or di-1-4C-alkylaminocarbonyl, 1-4C-alkylcarbonylamino, 1-4C-alkylcarbonyloxy, 1-4C-alkylsulfonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, phenylsulfonylamino, phenylsulfonylamino substituted in the phenyl moiety by R10 and/or R11, benzylsulfonylamino or benzylsulfonylamino substituted in the phenyl moiety by R12 and/or R13,
or
R4 is halogen, hydroxyl, nitro, trifluoromethyl, carboxyl, 1-4C-alkyl, 1-4C-alkoxy, 1-4C-alkoxycarbonyl, amino, mono- or di-1-4C-alkylamino, aminocarbonyl, mono- or di-1-4C-alkylaminocarbonyl, 1-4C-alkylcarbonylamino, 1-4C-alkylcarbonyloxy, 1-4C-alkylsulfonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, phenylsulfonylamino, phenylsulfonylamino substituted in the phenyl moiety by R10 and/or R11, benzylsulfonylamino or benzylsulfonylamino substituted in the phenyl moiety by R12 and/or R13, and
R5 is 1-4C-alkoxy which is completely or predominantly substituted by fluorine,
R6 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R7 is halogen, 1-4C-alkyl or 1-4C-alkoxy,
R8 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R9 is halogen, 1-4C-alkyl or 1-4C-alkoxy,
R10 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R11 is halogen, 1-4C-alkyl or 1-4C-alkoxy,
R12 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R13 is halogen, 1-4C-alkyl or 1-4C-alkoxy,
the salts of these compounds, as well as the N-oxides, enantiomers and tautomers of these compounds and their salts.

2. Compounds of formula 1 according to claim 1 in which
R1 is hydrogen, 1-2C-alkyl, phenyl, phenylethyl or phenylpropyl,
R2 is 1-hydroxy-2-4C-alkyl, 1-4C-alkylcarbonyl or 1-(acetyloxy)-2-4C-alkyl,
R3 is hydrogen, Arylbutyl, Heteroarylbutyl, Arylpropyl, Heteroarylpropyl, Arylethyl or Heteroarylethyl,
wherein
Aryl is phenyl or naphthalenyl,
Heteroaryl is pyridinyl, pyrimidinyl, thiofuranyl, indolyl or furanyl, and in which either
R4 is 1-4C-alkoxy which is completely or predominantly substituted by fluorine and
R5 is halogen, hydroxyl, nitro, trifluoromethyl, 1-4C-alkyl, 1-4C-alkoxy, 1-4C-alkoxy which is completely or predominantly substituted by fluorine, amino, mono- or di-1-4C-alkylamino, 1-4C-alkylcarbonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9,
or
R4 is halogen, hydroxyl, nitro, trifluoromethyl, 1-4C-alkyl, 1-4C-alkoxy, amino, mono- or d-1-4C-alkylamino, 1-4C-alkylcarbonylamino, phenylcarbonylamino, phenylcarbonylamino substituted in the phenyl moiety by R6 and/or R7, benzylcarbonylamino, benzylcarbonylamino substituted in the phenyl moiety by R8 and/or R9, and
R5 is 1-4C-alkoxy which is completely or predominantly substituted by fluorine,
R6 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R7 is halogen, 1-4C-alkyl or 1-4C-alkoxy,
R8 is halogen, hydroxyl, cyano, 1-4C-alkyl, trifluoromethyl, 1-4C-alkoxy, 1-4C-alkoxy which is completely or predominantly substituted by fluorine, carboxyl, 1-4C-alkoxycarbonyl, nitro, amino, mono- or di-1-4C-alkylamino, aminocarbonyl, aminosulfonyl, mono- or di-1-4C-alkylaminocarbonyl, mono- or di-1-4C-alkylaminosulfonyl, 1-4C-alkylcarbonylamino or 1-4C-alkylcarbonyloxy,
R9 is halogen, 1-4C-alkyl or 1-4C-alkoxy,
the salts of these compounds, as well as the N-oxides, enantiomers and tautomers of these compounds and their salts.

3. Compounds of formula 1 according to claim 1 in which
R1 is hydrogen, methyl, phenyl, phenylethyl or phenylpropyl,
R2 is 1-hydroxyethyl, acetyl or 1-(acetyloxy)ethyl,
R3 is hydrogen or phenylpropyl,
and in which either
R4 is difluoromethoxy and
R5 is nitro, amino, methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
or
R4 is chlorine or methoxy and
R5 is difluoromethoxy,
the salts of these compounds, as well as the N-oxides, enantiomers and tautomers of these compounds and their salts.

4. Compounds of formula 1 according to claim 1 in which
R1 is hydrogen or methyl,
R2 is 1-hydroxyethyl, acetyl or 1-(acetyloxy)ethyl,
R3 is phenylpropyl,
and in which either
R4 is difluoromethoxy and
R5 is methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
or
R4 is chlorine or methoxy and
R5 is difluoromethoxy,
the salts of these compounds, as well as the N-oxides, enantiomers and tautomers of these compounds and their salts.

5. Compounds of formula 1 according to claim 1 in which
R1 is hydrogen or methyl,
R2 is 1-hydroxyethyl, acetyl or 1-(acetyloxy)ethyl,
R3 is phenylpropyl,
and in which either
R4 is difluoromethoxy and
R5 is methoxy, 4-methoxyphenylmethylcarbonylamino, 4-methoxycarbonylphenylcarbonylamino, 4-dipropylaminosulfonylphenylcarbonylamino, 3-chloro-4-fluorophenylcarbonylamino or 3-fluoro-4-methylphenylcarbonylamino,
or
R4 is methoxy and
R5 is difluoromethoxy,
the salts of these compounds, as well as the N-oxides, enantiomers and tautomers of these compounds and their salts.

6. Compounds of formula 1 according to claim 1 for the treatment of diseases.

7. Pharmaceutical composition containing one or more compounds of formula 1 according to claim 1 together with the usual pharmaceutical auxiliaries and/or excipients.

8. Use of compounds of formula 1 according to claim 1 for the production of pharmaceutical compositions for the treatment of conditions of pathologically enhanced endothelial activity and impaired endothelial barrier function such as septic shock and vascular edema.

9. Use of compounds of formula 1 according to claim 1 for the production of pharmaceutical compositions for the treatment of (1) conditions associated with pathologically enhanced neoangiogenesis such as all kinds of tumors (benign or malignant) or (2) all kinds of inflammatory diseases associated with neoangiogenesis such as disorders of the arthritis type.

10. A method for treating conditions of pathologically enhanced endothelial activity and impaired endothelial barrier function such as septic shock and vascular edema in a patient in need thereof, comprising administering to said patient a therapeutically effective amount of a compound of formula 1 as claimed in claim 1.

11. A method for treating (1) conditions associated with pathologically enhanced neoangiogenesis such as all kinds of tumors (benign or malignant) or (2) all kinds of inflammatory diseases associated with neoangiogenesis such as disorders of the arthritis type in a patient in need thereof, comprising administering to said patient a therapeutically effective amount of a compound of formula 1 as claimed in claim 1.
